(19) United States
(12) Patent Application Publication
(10) Pub. No.: US 2003/0175217 A1
(43) Pub. Date: Sep. 18, 2003

(54) Thixotropic oral and dental care agents

(76) Inventors: Christian Kropf, Hilden (DE); Ulrike Brueninghaus, Monheim (DE)

Correspondence Address: HENKEL CORPORATION, 2500 RENAISSANCE BLVD, STE 200, GULPH MILLS, PA, 19406, US

(21) Appl. No.: 10/297,842

(22) PCT Filed: Jun. 7, 2001
(86) PCT No.: PCT/EP01/06425

(30) Foreign Application Priority Data
Jun. 16, 2000 (DE) 100289746

(51) Int. Cl. A61K 7/16

(52) U.S. Cl. 424/49

(57) ABSTRACT

The invention relates to thixotropic oral and dental care agents containing a content of one or more nanoparticular inorganic compounds selected from the group consisting of metal oxides, metal oxide hydrates, metal hydroxides, metal carbonates, metal phosphates and of metal silicates. The agents are suited, for example, as liquid-dosable or sprayable mouthwashes.

The invention relates to thixotropic oral and dental care compositions having a content of one or more nanoparticulate inorganic compounds from the group of metal oxides, oxide hydrates, hydroxides, carbonates, phosphates and silicates.

Besides tooth-pastes, in oral and dental care there is frequently use of liquid preparations with which the cleaning and care effects are achieved by contact of the preparation with the oral cavity without the need for an additional aid such as a toothbrush. These preparations, which are normally referred to as mouthwashes, provide in this way a particular convenience of use and moreover ensure that even regions of the oral cavity which are difficult to access, especially on the teeth, come into contact with the preparation.

Whereas mouthwashes have in the past been used merely for freshening the mouth and throat and for improving mouth odor, they now undertake tasks such as, for example, a partial plaque-dissolving effect, and they are used as carriers of anticaries agents or antibacterial agents for controlling plaque. Mouthwashes are made available as low-viscosity aqueous or hydroalcoholic formulations or as concentrates which can be diluted with water. Although application in the form of a low-viscosity preparation on the one hand achieves easy distribution and good contact with the oral cavity, a disadvantage in certain cases is the short contact time between the mouthwash and, in particular, the teeth. Intensive contact takes place only during the process of rinsing the mouth, and as soon as this ceases, only a thin film of the mouthwash remains on the teeth. This contact is too short and insufficiently intensive for certain agents employed in oral and dental care, so that such agents cannot be employed in mouthwashes or are able to display an only unsatisfactory, or no, effect. One example thereof comprises remineralizing agents which must initially become attached to the surface of the teeth, and subsequently contribute via biomineralization to repair and buildup of enamel.

A further disadvantage of conventional mouthwashes is that, owing to their low viscosity, it is very difficult to incorporate substances of low solubility stably into the formulations, and in this case there are frequently signs of sedimentation and thus unsatisfactory storage stabilities of such products.

U.S. Pat. No. 5 455 023 describes mouthwashes into which it is intended, because of its positive properties, e.g. as deodorant and buffer substance, to incorporate a particularly high concentration of sodium bicarbonate, which can be stably formulated only in low concentration in conventional mouthwashes. This object was achieved according to the invention by incorporating the sodium bicarbonate in the form of particles having an average particle diameter of from 500 to 5 000 nm. Optional addition of polymeric thickeners is described for additional stabilization of the formulations, and a further effect described therefore is an extended duration of action of the mouthwash residues. The preparations can be adjusted to a high viscosity with thickeners and then display thixotropic properties.

JP-A 09241152 describes chitin- or chitosan-containing thixotropic oil-in-water emulsions which are used for antiinflammatory compositions in the oral sector. Because of the thixotropic properties, a longer residence time at the site of action is said to be achieved. However, the skilled worker is aware that the formulation of chitin- and chitosan-containing compositions is, because of the problematic solubility of these biopolymers, associated with considerable difficulties and is possible only within ranges with narrow limits, especially in the case of alcohol-containing formulations.

The thickeners and thixotropes known in cosmetics have properties which are not advantageous for use in mouthwashes. Thus, in some cases considerable amounts of these substances are required to achieve the desired consistency of the thixotropic formulation, and in some cases the mechanical energy necessary for liquefying the formulation, for example by shaking, is too great and unacceptable for the user on use. However, a particular problem is that the time taken for the liquefaction and, on the other hand, the resolidification of the thixotropic formulation is too long. No satisfactory solutions are known from the prior art in particular for using thixotropic formulations in sprayable products.

It was an object of the present invention to provide a thixotropic oral and dental care composition which, in the resting state, is in the form of a gel with yield point, can be reversibly liquefied under use conditions relevant to the user, for example by shaking before use or by spraying, and forms a gel again after only a short time, for example within a few seconds or even immediately after the action of shear forces end.

It has been found, surprisingly, that certain inorganic substances in nanoparticulate form are particularly suitable for imparting thixotropy to oral and dental care products such as, for example, mouthwashes.

The invention relates to a thixotropic oral and dental care composition which comprises one or more nanoparticulate inorganic compounds from the group of metal oxides, oxide hydrates, hydroxides, carbonates, phosphates and silicates.

Preferred metals for the purposes of the invention are alkali metals, calcium, magnesium, aluminum, titanium, zircon and zinc, with particular preference for magnesium.

It is self-evident for the purposes of the invention that the compositions may also contain mixed inorganic compounds such as, for example, basic aluminum magnesium carbonates of the hydrotalcite type.

It is further preferred for the nanoparticulate inorganic compounds present in the compositions of the invention to have low solubility in water. Compounds of low solubility in water are intended to mean those having a solubility of less than 1 g/l and preferably of less than 1 mg/l in water at 20° C.

The average particle size of the nanoparticulate compounds is normally from 1 to 200 nm, preferably 5 to 100 nm, and particularly preferably 10 to 50 nm, the value referring to the particle diameter in the longitudinal direction, i.e. in the direction of the largest dimension of the particles.

The content of one or more nanoparticulate inorganic compounds from the group of metal oxides, oxide hydrates, hydroxides, carbonates, phosphates and silicates is normally from 0.1 to 20% by weight, preferably 0.2 to 10% by weight and particularly preferably 0.5 to 5% by weight, where the percent data are, in each case as total of the weight of the nanoparticulate inorganic compounds, based on the total weight of the compositions.

Suitable nanoparticulate oxides are, for example, magnesium oxide, aluminum oxide (Al₂O₃), titanium dioxide, zircon dioxide and zinc oxide, and silicon dioxide. A suitable nanoparticulate oxide hydrate is, for example, aluminum oxide hydrate (boehmite).

Suitable nanoparticulate silicates are, for example, aluminosilicates such as zeolites, and magnesium silicates. Preferred silicates are the sheet silicates (phyllosilicates), especially bentonites (contain as principal minerals smectites, especially montmorillonite), montmorillonites (Al₂[(OH)₂/Si₄O₁₀]·nH₂O and Al₂O₃·4SiO₂·H₂O·nH₂O, clay mineral belonging to the dioctahedral (mica) smectites), kaolinite (Al₂[(OH)₄/Si₂O₅] and Al₂O₃·2SiO₂·2H₂O, triclinic bilayer clay mineral (1:1 phyllosilicate)), talc (hydrated magnesium silicate of composition Mg₃[(OH)₂/Si₄O₁₀] or 3MgO·4SiO₂·H₂O) and, particularly preferably, hectorites (e.g. M⁺_{0.3}(Mg_{2.7}Li_{0.3}) [Si₄O₁₀(OH)₂], M⁺ usually=Na⁺, monoclinic clay mineral belonging to the smectites and similar to montmorillonite).

A preferred carbonate is hydrotalcite (International nonproprietary name for dialuminum hexamagnesium carbonate hexadecahydroxide tetrahydrate, Al₂Mg₅(OH)₁₆CO₃·4H₂O).

Likewise preferred according to the invention is nanoparticulate boehmite (ALO(OH), aluminum oxide hydrate) which is obtainable for example under the proprietary names Disperal® Sol P3 and Disperal® Sol P2 from Condea.

Nanoparticulate oxides, oxide hydrates or hydroxides can be prepared by known processes, e.g. as disclosed in EP-A-0 711 217 (Nanophase Technologies Corp.). Oxide hydrates and hydroxides in very fine distribution can also be obtained by hydrolysis of organometallic compounds.

In a particular embodiment of the invention there is use of nanoparticulate inorganic compounds with a specific surface area of more than 200 m²/g. A preferred nanoparticulate compound of this type is magnesium silicate of the sheet silicate type with a specific surface area of from 200 to 500 m²/g, in particular 300 to 400 m²/g. This material can be obtained in large quantities at reasonable cost. The product is available under the proprietary names Optigel® SH (Sutd-Chemie AG) and Laponite® XLG (Laporte Ltd.).

In a further particular embodiment of the invention, the nanoparticulate inorganic compounds are modified with one or more surface-modifying agents. Modification means the covering of the surface of the particles with organic compounds which interact via chemical bonds or physical forces with the surface of the particles.

Suitable surface-modifying agents for the nanoparticles are all monobasic and polybasic carboxylic acids and hydroxy carboxylic acids having 2 to 18 C atoms, that is to say, for example, acetic acid, propionic acid, oxalic acid, glutaric acid, maleic acid, succinic acid, phthalic acid, adipic acid, suberic acid, palmitic acid and stearic acid. Suitable and preferred are the hydroxy carboxylic acids and fruit acids such as, for example, glycolic acid, lactic acid, citric acid, malic acid, tartaric acid and gluconic acid. It is particularly preferred to employ as carboxylic acid a hydroxy carboxylic acid from the group of lactic acid, citric acid, malic acid and tartaric acid.

The surface modification of the inorganic nanoparticles preferably takes place by treatment with an aqueous solution of a carboxylic or hydroxy carboxylic acid in such a way that the nanoparticles are treated with a solution of from 0.05 to 0.5 mol of the carboxylic acid per mole of the nanoparticulate inorganic compound. This treatment preferably takes place over a period of from 1 to 24 hours at a temperature of at least 20° C., but preferably at the boiling point of water under atmospheric pressure (100° C.). If pressure is applied, the treatment can also take place at temperatures above 100° C. in a correspondingly shorter time.

The surface of the nanoparticles is modified by the treatment with the carboxylic acids or hydroxy carboxylic acids. It is assumed that the carboxylic acids or hydroxy carboxylic acids are bound in ester fashion to the surface of the nanoparticles.

The surface-modified nanoparticles are isolated from the reaction mixture, preferably by dehydration. The dispersion is for this purpose preferably subjected to a freeze drying. This entails the solvent being sublimed off at low temperature under high vacuum.

Inorganic nanoparticles modified by this process contain between 1 and 30% by weight, preferably between 5 and 20% by weight, of the organic surface-modifying agent based on the total weight of the surface-modified inorganic nanoparticles.

For surface modification of the nanoparticles it is also possible to employ functional silanes of the type (OR)₄₋ₙSiRₙ (R=org. radicals having functional groups such as hydroxyl, carboxyl, ester, amine, epoxy, etc.), quaternary ammonium compounds, phosphonic acids or amino acids. Depending on the polarity of the modifying agent, the modification described above will be carried out in water or in organic solvents (alcohols, ethers, ketones, hydrocarbons, etc.), choosing the reaction conditions in analogy to those in water. Sheet silicates such as, for example, hectorites can also be subjected to an ion exchange, incorporating cations such as, for example, quaternary ammonium compounds between the sheets of the material. Examples of further suitable surface-modifying agents are gelatin, starch, dextrin, dextran, pectin, gum arabic, casein, gums, polyvinyl alcohols, polyethylene glycols, polyvinylpyrrolidone, polyvinylbutyrals, methylcellulose, carboxymethylcellulose, hydroxypropylcellulose, or else surfactants and emulsifiers such as, for example, fatty alcohol polyglycol ethers, fatty alcohol polyglycosides, fatty acid alkanolamides, glycerol esters, sorbitan esters or alkoxylated esters and derivatives thereof.

The compositions of the invention may additionally comprise ingredients like those usual for oral and dental care compositions, such as, for example, surfactants, flavorings, solubilizers, oral-hygiene agents, colors and opacifying agents.

Surfactants which can be employed are anionic, cationic, zwitterionic, ampholytic and nonionic surface-active substances with a solubility in water of at least 1% by weight (20° C.), in amounts of from 0.1 to 5% by weight, preferably of from 0.1 to 1% by weight.

Preferred surface-active substances in this connection are substances which comprise a lipophilic, linear alkyl or acyl group having 10 to 22 C atoms and a water-solubilizing ionic group, e.g. a sulfate, sulfonate, phosphate, carboxylate or, for example, a trimethylammonium group or an acetobetaine group or a nonionic polyhydroxylalkyl or polyoxyethylene group. Examples of suitable ionic surfactants are, for example, sodium lauryl sulfate, sodium lauroyl-isethionate, cetyltrimethylammonium chloride, lauryl-trimethylammonium acetobetaine, lauroylamidopropyl-dimethylammonium acetobetaine. However, nonionic surface-active substances are preferably present, and examples which are mentioned are adducts of ethylene oxide with fatty alcohols, with fatty acids, with fatty acid monoglycerides, with sorbitan fatty acid esters, with propylene glycol mono-fatty acid esters or with methylglycoside mono-fatty acid esters. Further suitable and preferred nonionic surfactants are alkyl (oligo)glycosides.

Concerning the glycoside residue, both monoglycosides (x=1) in which a hexose residue is glycosidically linked to a fatty alcohol having 8 to 16 C atoms, and oligomeric glycosides having a degree of oligomerization x of up to 10 are suitable. The degree of oligomerization in this connection is a statistical average based on a distribution of homologs usual for such industrial products.

Suitable and preferred as alkyl (oligo)glycoside is an alkyl (oligo)glucoside of the formula RO(C₆H₁₀O)ₓ—H, in which R is an alkyl group having 12 to 14 C atoms, and x has an average value of from 1 to 4.

Apart from the alkyl glucoside surfactants mentioned, it is also possible for other nonionic, ampholytic and cationic surfactants to be present. A nonionic solubilizer from the group of surface-active compounds may be necessary in particular for solublizing the aromatic oils which are usually insoluble in water. Examples particularly suitable for this purpose are ethbxylated fatty acid glycerides, ethoxylated fatty acid sorbitan partial esters or fatty acid partial esters of glycerol ethoxylates or sorbitan ethoxylates. Solubilizers from the group of ethoxylated fatty acid glycerides comprise in particular adducts of 20 to 60 mol of ethylene oxide with mono- and diglycerides of linear fatty acids having 12 to 18 C atoms or with triglycerides of hydroxy fatty acids such as oxystearic acid or ricinoleic acid. Further suitable solubilizers are ethoxylated fatty acid sorbitan partial esters; these are preferably adducts of 20 to 60 mol of ethylene oxide with sorbitan monoesters and sorbitan diesters of fatty acids having 12 to 18 C atoms. Solubilizers which are likewise suitable are fatty acid partial esters of glycerol ethoxylates or sorbitan ethoxylates; these are preferably monoesters and diesters of C₁₂-C₁₈-fatty acids and adducts of 20 to 60 mol of ethylene oxide with 1 mol of glycerol or with 1 mol of sorbitol.

The compositions of the invention comprise as solubilizers for aromatic oils which are present where appropriate preferably adducts of 20 to 60 mol of ethylene oxide with hardened or unhardened castor oil (i.e. with oxystearic acid triglyceride or ricinoleic acid triglyceride), with glycerol monostearate and/or distearate or with sorbitan monostearate and/or distearate.

Flavorings which may be present are, for example, sweeteners and/or aromatic oils. Suitable aromatic oils are all natural and synthetic aromas in use for oral and dental care compositions. Natural aromas can be used both in the form of the essential oils isolated from the herbs and of the individual components isolated therefrom. At least one aromatic oil from the group of peppermint oil, spearmint oil, anise oil, star-anise oil, caraway oil, eucalyptus oil, fennel oil, cinnamon oil, clove oil, geranium oil, sage oil, allspice oil, thyme oil, marjoram oil, basil oil, citrus oil, wintergreen oil or one or more synthetically produced components, isolated therefrom, of these oils should preferably be present. The principal components of said oils are, for example, menthol, carvone, anethole, cineole, eugenol, cinnamaldehyde, caryophyllene, geraniol, citronellol, linalool, salvene, thymol, terpinene, terpinol, methyl chavicol and methyl salicylate. Further suitable aromas are, for example, menthyl acetate, vanillin, ionones, linalyl acetate, rhodinol and piperitone.

Examples of suitable sweeteners are saccharin sodium, sodium cyclamate, acesulfame K, aspartame, lactose, maltose, fructose, glycerol, sorbitol, mannitol or xylitol.

Finally, the compositions of the invention may comprise oral-hygiene and therapeutic agents such as, for example,

caries-inhibiting fluorine compounds, e.g. sodium fluoride, tin fluoride, sodium monofluorophosphate or amine fluoride

antiplaque agents, e.g. organophosphonates, sodium pyrophosphate, sodium tripolyphosphate

wound-healing and antiinflammatory substances such as, for example, allantoin, urea, azulene or camomile extract,

antibacterial, plaque-inhibiting substances such as, for example, chlorhexidine or triclosan.

In a preferred embodiment of the invention, the compositions of the invention comprise one or more remineralizing agents. Examples of suitable remineralizing agents are fluorides such as cetylamine hydrofluoride and calcium salts.

The remineralizing agents are particularly preferably selected from the group formed by phosphates, fluorides and fluorophosphates of calcium which have low solubility in water, in particular those having an average particle diameter in the range from 5 to 300 nm, which may additionally be coated with surfactants or protective colloids or be in the form of composites with protein components. Examples of such calcium salts which are particularly suitable for the purposes of the present invention are the nanoparticulate calcium salts described in the DE patent application 19858662.0, and the composite materials composed of calcium salts and protein components described in DE patent application 19930335.5. It is particularly preferred for the purposes of the present invention for the compositions of the invention to comprise nanoparticulate apatite, hydroxyapatite or fluoroapatite as remineralizing agent.

The remineralizing agents are preferably employed in the form of nanoparticles since in this case, because of the large specific surface area of the nanoparticles, there is particularly good adhesion to the natural material of the teeth and the fine particles remain adherent to the enamel even after rinsing out. They penetrate preferentially into small irregularities such as cracks or fissures and bring about rapid remineralization there.

The content of remineralizing agents in the compositions of the invention is normally from 0.1 to 10% by weight and preferably from 0.5 to 5% by weight, based on the total weight of the compositions.

The preparations of the invention may additionally comprise between 0.5 and 15% by weight of ethanol.

The compositions of the invention are particularly suitable as mouthrinses or mouthwashes. The invention therefore also relates to the use of the compositions of the invention as mouthrinse or mouthwash.

The compositions of the invention can be sprayed—both with a spray pump and as aerosol. The invention therefore also relates to the use of the compositions of the invention as sprayable oral and dental care composition, especially as sprayable mouthwash.

In the resting state, the compositions of the invention form a solid gel, and only through the movements in the mouth does liquefaction occur to give a watery-thin system. Solidification occurs anew immediately without rinsing movements. The active ingredients thereby remain on the teeth for longer and may bond by adhesion or be taken up. This is particularly important for example for agents for remineralization of the teeth and for antibacterial agents.

The nanoparticulate inorganic compounds employed in the compositions of the invention have a very efficient thixotropic effect, i.e. even with use of relatively small amounts and at little cost it is possible to produce compositions with very pronounced thixotropy.

The thixotropic compositions of the invention make it possible for even particulate substances of low solubility to be incorporated stably into the formulations, and there are no manifestations of sedimentation nor unsatisfactory storage stabilities of such compositions.

It is particularly advantageous that, on the one hand, the liquefaction of the thixotropic compositions after introduction of mechanical shear energy, and, on the other hand, resolidification in the resting state takes place very rapidly, for example within a few seconds or even immediately after the action of the shear forces ends. This is very particularly valuable for the use of such thixotropic compositions in sprayable products. Thus, the compositions of the invention can be applied for example with the aid of pump atomizers, in which case the liquefaction can take place soley through the actuation of the pump atomizer and without previous shaking of the atomizer. As soon as the sprayed mist of the composition has settled on the surface of the teeth, it solidifies.

A further advantage of the nanoparticulate inorganic compounds employed for imparting thixotropy to the compositions of the invention is that they show a substantially neutral behavior in the composition, that is to say do not have an adverse effect on the other ingredients present in the composition.

An additional advantage of the compositions of the invention is their transparency, which is not impaired by the nanoparticulate inorganic compounds.

The compositions of the invention are produced by mixing the ingredients, where appropriate with use of ultrasound.

The following examples are intended to illustrate the present invention without restricting it. All percentage contents are based on the total weight of the preparations unless otherwise indicated.

### EXAMPLES

### Example 1

Mouth Gel

| Ingredient | Content in % by weight |
|---|---|
| Ethanol | 2.4 |
| Sorbitol 70% | 4 |
| Cremophor RH 60 | 2 |
| Chlorhexidine | 0.2 |
| Plantacare 1200 | 1 |
| Aroma | 0.2 |
| Color | 0.001 |
| Hectorite (Laponite XLG) | 4 |
| Nanoparticulate hydroxyapatite | 2 |
| Water | to 100 |

Cremophor RH 60: hydrogenated castor oil with 60 EO Plantacare 1200: C12-C16-alkyl glycoside Nanoparticulate hydroxyapatite: prepared as in example 1.1. of the patent application DE 19858662.0, replacing Plantacare 1200 by Cremophor RH 60; the content indicated in the above table relates to the anhydrous hydroxyapatite, and the Cremophor RH 60 included as a result of the preparation is accounted for separately in the table.

### Example 2

Sprayable Mouth Gel

| Ingredient | Content in % by weight |
|---|---|
| Ethanol | 5 |
| Sorbitol 70% | 3 |
| Chlorhexidine | 0.2 |
| APG 220 UPW | 0.5 |
| Cremophor RH 60 | 0.1 |
| Aroma | 0.001 |
| Color | 0.001 |
| Hectorite (Laponite XLG) | 2 |
| Nanoparticulate hydroxyapatite | 1 |
| Citric acid | 0.05 |
| Water | to 100 |

APG 220 UPW: C8-10 alkyl 1,5-glucoside, AS content 62-65%

The gel has a pH of 7.3 and, in the resting state, has a sliceable consistency.

1. A thixotropic oral and dental care composition, characterized in that it comprises one or more nanoparticulate inorganic compounds from the group of metal oxides, oxide hydrates, hydroxides, carbonates, phosphates and silicates.

2. The composition as claimed in claim 1, characterized in that the metal is selected from the group formed by alkali metals, calcium, magnesium, aluminum, titanium, zircon and zinc.

3. The composition as claimed in either of claims 1 or 2, characterized in that the average particle size of the nanoparticulate compounds is from 1 to 200 nm, preferably from 5 to 100 nm, particularly preferably 10 to 50 nm.

4. The composition as claimed in any of claims 1 to 3, characterized in that it contains one or more nanoparticulate compounds in an amount totalling from 0.1 to 20% by weight, preferably 0.2 to 10% by weight, particularly preferably 0.5 to 5% by weight.

5. The composition as claimed in any of claims 1 to 4, characterized in that it comprises hectorite.

6. The composition as claimed in any of claims 1 to 5, characterized in that the nanoparticulate inorganic compound is modified with at least one surface-modifying agent.

7. The composition as claimed in any of claims 1 to 6, characterized in that it comprises at least one remineralizing agent.

8. The composition as claimed in claim 7, characterized in that the remineralizing agent is selected from the group formed by phosphates, fluorides and fluorophosphates of calcium which have low solubility in water, in particular having an average particle diameter in the range from 5 to 300 nm.

9. The composition as claimed in either of claims 7 or 8, characterized in that the remineralizing agent is present in an amount of from 0.1 to 10% by weight, preferably 0.5 to 5% by weight, based on the total weight of the composition.

10. The use of a composition as claimed in any of claims 1 to 9 as mouthwash.

11. The use of a composition as claimed in any of claims 1 to 9 as sprayable oral and dental care composition, in particular as sprayable mouthwash.
